(19) United States
(12) Patent Application Publication
(10) Pub. No.: US 2004/0176853 A1
(43) Pub. Date: Sep. 9, 2004

(54) Apparatus and method for spinal fusion using posteriorly implanted devices

(76) Inventors: Andrew R. Sennett, Hanover, MA (US); Roland F. Gatturna, Winthrop, MA (US); Gary L. Lowery, Phoenix, AZ (US)

Correspondence Address: Burns & Levinson LLP, Suite 300, 1030 Fifteenth Street, N.W., Washington, DC, 20005-1501, US

(21) Appl. No.: 10/379,609

(22) Filed: Mar. 5, 2003

(51) Int. Cl. A61F 2/44

(52) U.S. Cl. 623/17.16

(57) ABSTRACT

An apparatus and method for spinal interbody fusion is disclosed. This implant includes fasteners which firmly attach it to vertebrae adjacent to excised tissue so as to transmit tension and torsional loads to and from those vertebrae. The instruments and methods are particularly adapted for interbody fusion from a posterior approach to the spine. One instrument is a vertebral spreader that is able to create anterior lift to a fixed or variable angle. Another instrument is a tome for cutting rectangular grooves in bone and preparing end plate surfaces. A method contemplates the use of these instruments to prepare a disk space and the insertion of the implant.

### BACKGROUND OF INVENTION

1. Field of Invention

This invention relates generally to the treatment of injured, degenerated, or diseased tissue in the human spine, for example, damaged intervertebral discs and vertebrae. It further relates to the removal of damaged tissue and to the stabilization of the remaining spine by fusion to one another of at least two vertebrae adjacent or nearly adjacent to the space left by the surgical removal of tissue. More particularly, this invention relates to the implantation of devices that can be inserted from the patient's posterior, that is, from the back, to take the structural place of removed discs and vertebrae during healing while simultaneously sharing compressive loads. This invention further relates to the implantation of devices that do not interfere with the natural lordosis of the spinal column. More particularly, while aspects of the present invention may have other applications, the invention also provides instruments and techniques especially suited for interbody fusion from a generally posterior approach to the spine.

2. Background of the Invention

For many years a treatment, often a treatment of last resort, for serious back problems has been spinal fusion surgery. Disc surgery, for example, typically requires removal of a portion or all of an intervertebral disc. Such removal, of course, necessitates replacement of the structural contribution of the removed disc. The most common sites for such surgery, namely those locations where body weight most concentrates its load, are the lumbar discs in the L1-2, L2-3, L3-4, L4-5, and L5-S1 intervertebral spaces. In addition, other injuries and conditions, such as tumor of the spine, may require removal not only of the disc but of all or part of one or more vertebrae, creating an even greater need to replace the structural contribution of the removed tissue. Also, a number of degenerative diseases and other conditions such as scoliosis require correction of the relative orientation of vertebrae by surgery and fusion.

In current day practice, a surgeon will use one or more procedures currently known in the art to fuse remaining adjacent spinal vertebrae together in order to replace the structural contribution of the affected segment of the disc-vertebrae system. In general for spinal fusions a significant portion of the intervertebral disk and, if necessary, portions of vertebrae are removed and a stabilizing element, frequently including or composed entirely of bone graft material, is packed in the intervertebral space. In parallel with the bone graft material, typically additional external stabilizing instrumentation and devices are applied, in one method a series of pedicle screws and conformable metal rods. The purpose of these devices, among other things, is to prevent shifting and impingement of the vertebrae on the spinal nerve column. These bone graft implants and pedicle screws and rods, however, often do not provide enough stability to restrict relative motion between the two vertebrae while the bone grows together to fuse the adjacent vertebrae.

Various surgical methods have been devised for the implantation of fusion devices into the disk space. Both anterior and posterior approaches have been used for interbody fusions. The anterior approach requires the added costs and associated risks for a general surgeon and/or a vascular surgeon to open the patient's abdominal cavity in order for the back surgeon to operate on the spine from an anterior approach. As a result, many surgeons prefer a posterior approach.

The posterior surgical approach to the spine has often been used in the past. The primary difficulty of the posterior approach is that the spine surgeon must navigate past the spinal cord and subsidiary nerve structures. Also, unprotected drilling or trephining for implantation of cylindrical bone dowels carries risks to the patient.

U.S. Pat. No. 5,484,437 to Michelson discloses a technique and associated instrumentation for inserting a fusion device from a posterior surgical approach that provides greater protection for the surrounding tissues and neurological structures during the procedure. As described in more detail in the '437 patent, the surgical technique involves the use of a distractor having a penetrating portion that urges the vertebral bodies apart to facilitate the introduction of the necessary surgical instrumentation. The '437 patent also discloses a hollow sleeve having teeth at one end that are driven into the vertebrae adjacent the disc space created by the distractor. These teeth engage the vertebrae to maintain the disc space height during subsequent steps of the procedure following removal of the distractor. In accordance with one aspect of the '437 patent, a drill is passed through the hollow sleeve to remove portions of the disc material and vertebral bone to produce a prepared bore for insertion of the fusion device. The drill is then removed from the sleeve and the fusion device is positioned within the disc space using an insertion tool.

While the more recent techniques and instrumentation represent an advance over earlier surgical procedures for the preparation of the disc space and insertion of the fusion device, the need for improvement still remains. The present invention is directed to this need and provides convenient methods and instruments to insure safe and effective preparation of a disc space in conjunction with implant placement.

The restoration of normal anatomy is a basic principle of all orthopedic reconstructive surgery. Lordosis, which results in a pronounced forward curvature of the lumbar region of the spine, is a factor that needs to be taken into account in designing lumbar implants.

Therefore, there is a perceived need for a device which simultaneously and reliably attaches mechanically to the bony spinal segments on either side of the removed tissue so as to prevent relative motion in extension or torsion of the spinal segments during healing, provides spaces in which bone growth material can be placed to create or enhance fusion, and enables the new bony growth, and, in a gradually increasing manner if possible, shares the spinal compressive load with the bone growth material and the new growth so as to enhance bone growth and calcification. The needed device will usually require a modest taper to preserve natural lumbar spinal lordosis.

Thus, it is an object of the current invention to provide a stabilizing device for insertion in spaces created between vertebrae during spinal surgery. It is a further object to create a device implantable from the patient's posterior for stabilizing the spine by preventing or severely limiting relative motion between the involved vertebrae in tension (extension) and torsion loading during healing. It is a further object to provide a device which promotes growth of bone between vertebrae adjacent to the space left by the excised material by progressive sharing of the compressive load to the bone graft inserted in the space between the vertebrae. It is yet a further object to provide mechanical stability between adjacent vertebrae while bone grows and at the same time not diminish the natural lordosis of the lumbar spine. It is a further object to provide instrumentation that provides adequate protection for the sensitive vessels and neurological structures adjacent to the operating field. It is a further object to provide instrumentation that create a significant bed of bleeding bone while also preserving endplate structure for strength. Another object of this invention is to provide a spreader instrument that provides sequential angular and translational distraction of the disk space from the posterior side to restore the natural height and angle of the disk space and to help facilitate insertion of the implant. It is yet another object of this invention for the implant to be capable of being fabricated from human bone allograft material.

### SUMMARY OF THE INVENTION

The invention disclosed here is a novel implant and associated instrumentation designed to achieve the foregoing objects. The design of the new implant for spinal surgery includes the possibility of fabricating the device from human bone allograft material and from biocompatible manmade materials. The design is also such that the implant seats firmly in and mechanically mates with and ultimately fastens to adjacent vertebrae and stabilizes the involved vertebrae in tension and in torsion. Either the implant can be tapered or the vertebrae can be cut so as to preserve the natural lordosis of the spine. This invention also includes instrumentation necessary to effectively and safely prepare the intervertebral space, angularly distract the vertebrae and insert the implants.

The implants generally have a rectangular geometry, although in some embodiments an annular geometry is preferred, with a lordotic slope of approximately ten degrees, such that the opposing anterior side is taller than the posterior side. The slope can be reversed for use in other portions of the spine for lumbar use where the curvature is opposite The implant also incorporates an anti-expulsion feature, such as notches or teeth on the top and bottom surfaces of the stabilizing fins. The implant may also contain opposing slots on either side to facilitate gripping with a bone holder instrument.

The attachment portions of the implant are stabilizing fins projecting inferiorly and superiorly from the central one third of the wedge shaped implant. These stabilizing fins help stabilize the disk space in torsion, and help maintain a stable host-graft interface for fusion. The top and bottom surfaces of the implants, including the fins themselves, have a typically ten-degree lordosis. Therefore the anterior portion of the implant is a taller dimension than the posterior portion. This creates a challenge in the placement of the implant from the posterior direction. Therefore, for the lumbar region, the implant has an aggressive lead chamfer designed to further distract the endplates, as necessary to facilitate placement.

In its most general form, the invention is an implant for mechanically attaching to the ends of and promoting bony fusion of at least two vertebrae adjacent to a space left by surgically removed spinal tissue, comprising a load-sharing body; said load-sharing body further comprising opposing rectangular fins on the top and bottom surfaces of the implant capable of mechanically anchoring the device to said adjacent vertebrae and thereby transmitting tensile and torsional loads to and from said adjacent vertebrae. In another embodiment, the vertebrae may be cut at such an angle so as to preserve the natural lordosis of the spine, i.e. 0-18 degrees, once the implant is inserted.

In another embodiment, the invention generally is an implant for mechanically attaching to the ends of and promoting bony fusion of at least two vertebrae adjacent to a space left by surgically removed spinal tissue, comprising a structure formed from a single piece of bone allograft material having a top and bottom, said top and bottom surfaces including a stabilizing fin for mechanically interlocking with channels cut into said adjacent vertebrae.

An important aspect in the implant procedure is the preparation of the space to receive the implant and the grooves for the rectangular stabilizing fins. A spacer/osteotome guide system is used which distracts the vertebrae and stabilizes them during preparation and acts as a guide for precise cutting. Special tomes are designed to precisely cut the rectangular channels and prepare the end plate surface. The spacer/osteotome guide is designed to avoid the nerve root and limit the depth of the cut for safety. The tomes also have depth stops which limit the depth of the cut for safety.

Another important aspect of the implant procedure is an instrument system to facilitate translational and angular distraction from within the disk space to achieve the quality of distraction currently only obtained by the anterior approach. This provides a highly significant benefit to the surgeon. A double action vertebral spreader is provided that will penetrate more deeply into the disk space to create anterior lift to a fixed or variable angle. The design will allow the surgeon to set the lordotic angle prior to distraction of the vertebral endplates.

### BRIEF DESCRIPTION OF THE DRAWINGS

FIG. 1A is an oblique view of the front and side of the spacer/osteotome guide part of the present invention;

FIG. 1B is an orthogonal view of the side of the spacer/osteotome guide;

FIG. 2 shows the osteotome in oblique view;

FIG. 3A is a top view of the implant;

FIG. 3B is a side view of the implant;

FIG. 3C is a posterior view of the implant;

FIG. 4A shows a posterior view of two vertebrae;

FIG. 4B shows a posterior view of two vertebrae with spacer/osteotome guides installed between the vertebrae;

FIG. 5A is an oblique view of the rear and side of the spacer/osteotome guide;

FIG. 5B is an oblique view of the front and side of the spacer/osteotome guide;

FIG. 5C shows a side view of the spacer/osteotome guide between two vertebrae;

FIG. 6A is a detailed oblique view of the dual box osteotome;

FIG. 6B shows the relationship of the dual box osteotome and the spacer/osteotome guide;

FIG. 7A shows a posterior view of two vertebrae in which channels have been cut in adjacent endplates;

FIG. 7B shows a posterior view of two vertebrae in which the implants have been installed;

FIG. 7C shows a side view of the two vertebrae in which an implant has been installed;

FIG. 8 is an oblique view of the vertebral body spreader;

FIG. 9A is a view of the vertebral body spreader in a closed mode;

FIG. 9B is a view of the vertebral body spreader with its jaws open at the lordotic angle;

FIG. 9C is a view of the vertebral body spreader in its fully open mode;

FIG. 10A is an oblique view of the rear and side of the spacer/osteotome guide;

FIG. 10B is an orthogonal view of the side of the spacer/osteotome guide;

FIG. 10C is an oblique view of the front and side of the spacer/osteotome guide part of the present invention;

FIG. 10D is a posterior view of the spacer/osteotome guide;

FIG. 10E is an oblique view of the front and side of the spacer/osteotome guide part of the present invention;

FIG. 10F is a posterior view of the spacer/osteotome guide;

FIG. 11A is a side view of the implant.

FIG. 11B is a side view of the implant.

### IDENTIFICATION OF ITEMS IN THE FIGURES

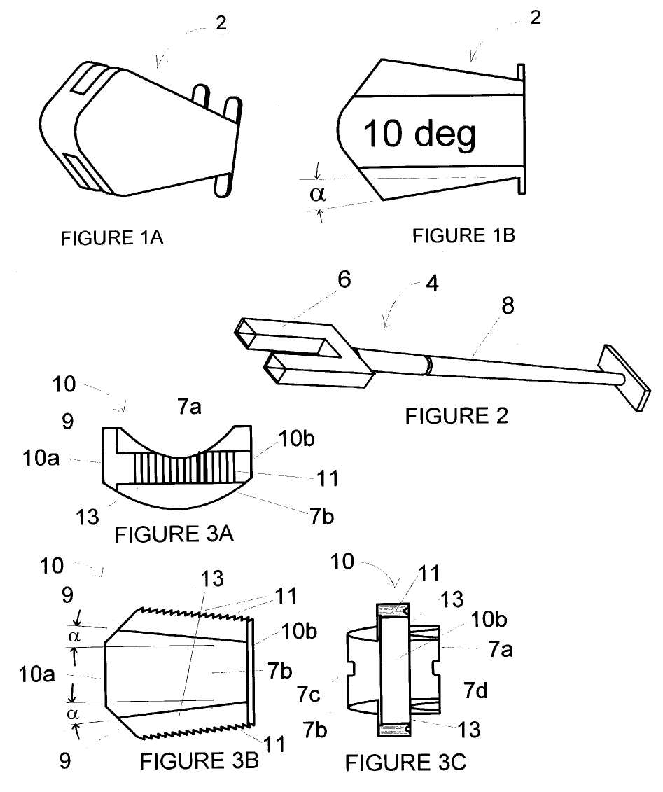

FIG. 1A

2—spacer/osteotome guide

FIG. 1B

2—spacer/osteotome guide

FIG. 2

4—osteotome

6—blades of osteotome

8—handle of osteotome

15—end of handle portion of dual box osteotome

FIG. 3A

7a,7b—curved sides of bone implant

9—chamfer

10—bone implant

11—locking teeth

13—fins on implant

FIG. 3B

7b—curved sides of bone implant

9—chamfer

10—bone implant

11—locking teeth

13—fins on implant

FIG. 3C

7b—curved sides of bone implant

7c,7d—opposing slots to facilitate gripping by a holding instrument

10—bone implant

11—locking teeth

13—fins on implant

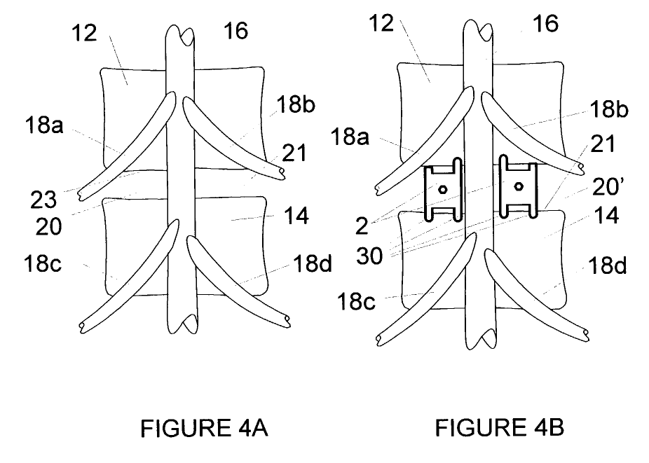

FIG. 4A

12—upper vertebral body

14—lower vertebral body

16—spinal cord

18a,18b,18c,18d—lateral nerves from spinal cord

20—intervertebral space

21—upper cortical endplate of lower vertebra

23—lower cortical endplate of upper vertebra

FIG. 4B

2—spacer/osteotome guide

12—upper vertebral body

14—lower vertebral body

16—spinal cord

18a,18b,18c,18d—lateral nerves from spinal cord

20′—intervertebral space

21—upper cortical endplate of lower vertebra

30—stop taps on spacer/osteotome guide

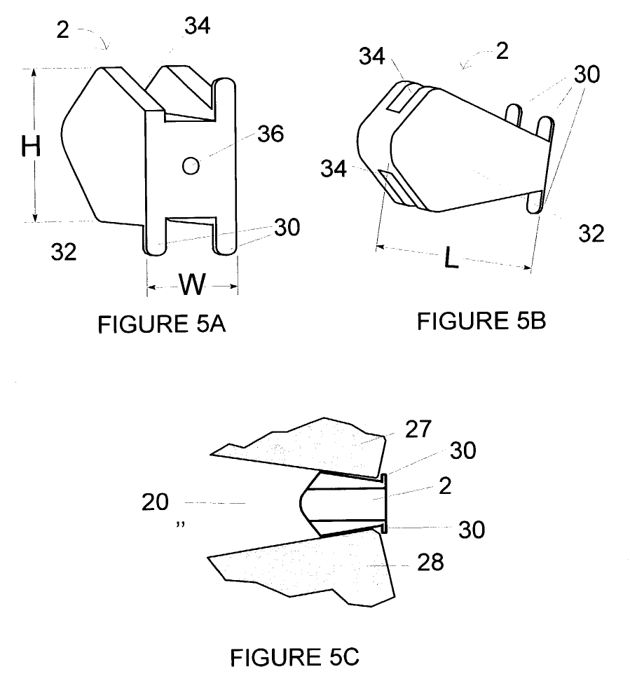

FIG. 5A

2—spacer/osteotome guide

30—stop tabs

32—angled side of spacer/osteotome guide

34—guide channels

36—hole that receives insertion handle

FIG. 5B

2—spacer/osteotome guide

30—stop tabs

32—angled side of spacer/osteotome guide

34—guide channels

FIG. 5C

2—spacer/osteotome guide

20″—intervertebral space

27—upper vertebral body

28—lower vertebral body

30—stop tabs

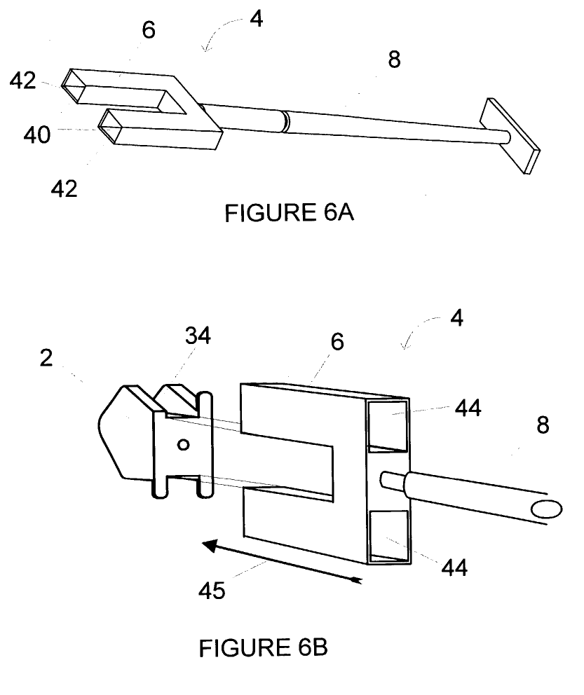

FIG. 6A

4—dual box osteotome

6—blades of osteotome

8—handle of osteotome

15—end of handle portion of dual box osteotome

40—front sides of osteotome blades

42—sharp cutting edges of osteotome

FIG. 6B

2—spacer/osteotome guide

4—dual box osteotome

6—blades of osteotome

8—handle of osteotome

34—osteotome guide channel

44—hollow centers of osteotome blades

45—arrow showing direction of cut

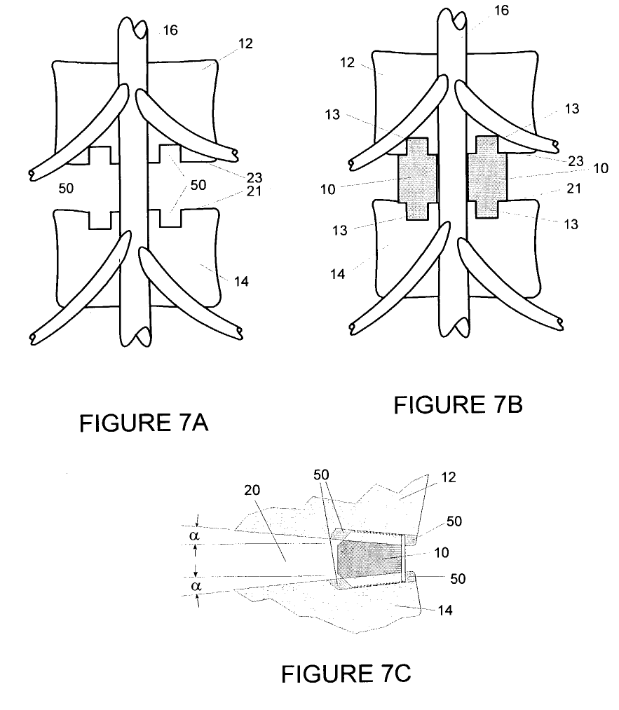

FIG. 7A

12—upper vertebra

14—lower vertebra

16—spinal cord

21—upper cortical endplate of lower vertebra

23—lower cortical endplate of upper vertebra

50—cut channels that receive implants

FIG. 7B

10—bone implants

12—upper vertebra

14—lower vertebra

16—spinal cord

21—upper cortical endplate of lower vertebra

23—lower cortical endplate of upper vertebra

50—cut channels that receive implants

FIG. 7C

10—bone implants

12—upper vertebra

14—lower vertebra

20—intervertebral space

50—cut channels that receive implants

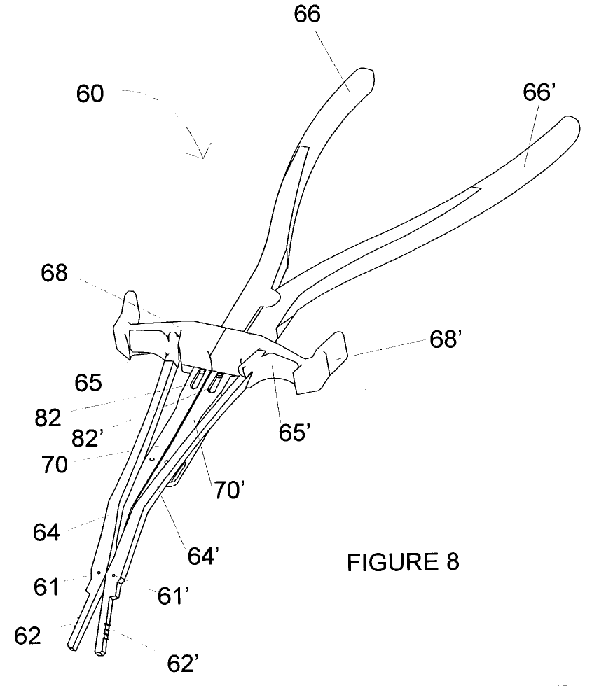

FIG. 8

60—vertebral body spreader tool

61,61′—pivot points, or hinge pins, of jaw actuation arms

62,62′—jaws

64,64′—jaw actuation arms

65,65′—catch mechanisms

66,66′—handles

68,68′—locking stops

70,70′—parallel rails

82, 82′—guide slots for sliding pivot arms

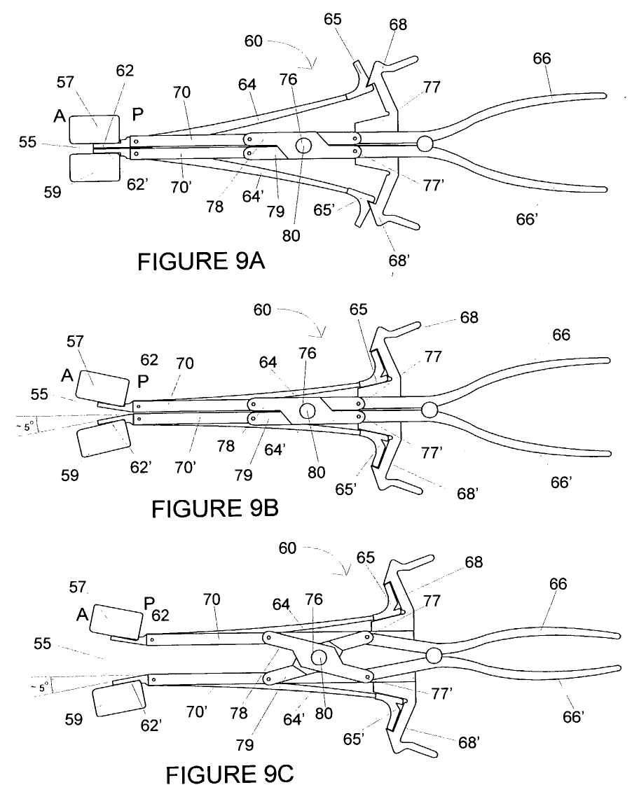

FIG. 9A

55—disc space

57—upper vertebral body

59—lower vertebral body

60—vertebral body spreader tool

62,62′—jaws

64,64′—jaw actuation arms

65,65′—catch mechanisms

66,66′—handles

68,68′—locking stops

70,70′—parallel rails

76,76′—crossing slider mechanism

77,77′—sliding pivot points

78—one arm of crossing slider

79—one arm of crossing slider

80—common pivot point of arms of crossing slider

FIG. 9B

55—disc space

57—upper vertebral body

59—lower vertebral body

60—vertebral body spreader tool

62,62′—jaws

64,64′—jaw actuation arms

65,65′—catch mechanisms

66,66′—handles

68,68′—locking stops

70,70′—parallel rails

76,76′—crossing slider mechanism

77,77′—sliding pivot points

78—one arm of crossing slider

79—one arm of crossing slider

80—common pivot point of arms of crossing slider

FIG. 9C

55—disc space

57—upper vertebral body

59—lower vertebral body

60—vertebral body spreader tool

62,62′—jaws

64,64′—jaw actuation arms

65,65′—catch mechanisms

66,66′—handles

68,68′—locking stops

70,70′—parallel rails

76,76′—crossing slider mechanism

77,77′—sliding pivot points

78—one arm of crossing slider

79—one arm of crossing slider

80—common pivot point of arms of crossing slider

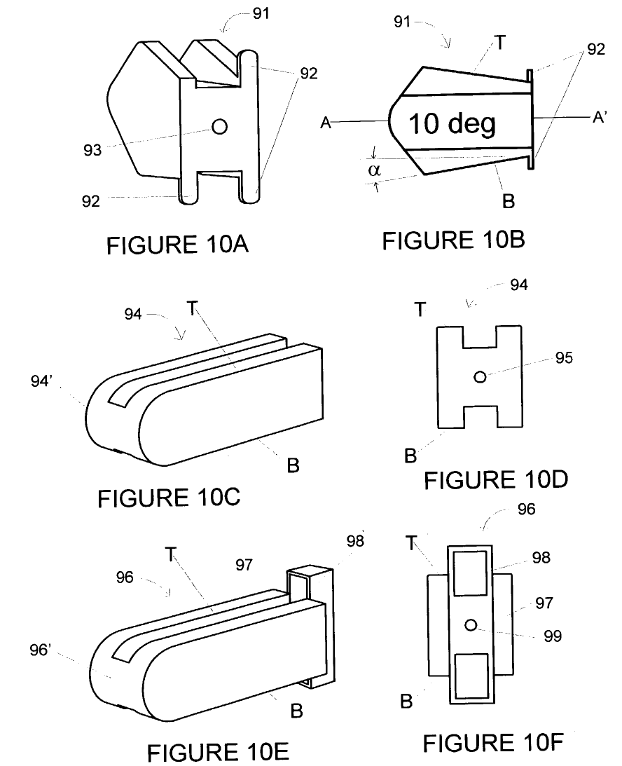

FIG. 10A

91—spacer/osteotome guide

92—stop tabs

93—hole

FIG. 10B

91—spacer/osteotome guide

92—stop tabs

FIG. 10C

94—spacer/osteotome guide

94′—blunt nose on anterior end of guide

FIG. 10D

94—spacer/osteotome guide

95—hole

FIG. 10E

96—spacer/osteotome guide

96′—blunt nose on anterior end of guide

97—main body of spacer

98—box guide for osteotome

FIG. 10F

96—spacer/osteotome guide

97—main body of spacer

98—box guide for osteotome

99—hole for detachable handle

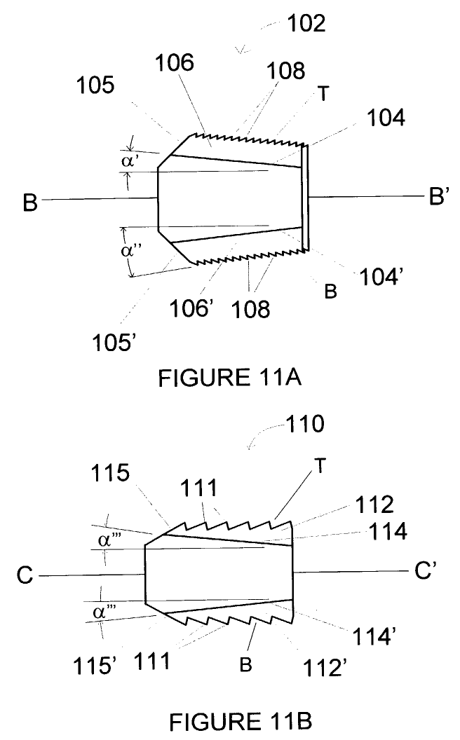

FIG. 11A

102—implant

104,104′—shelves

105,105′—chamfers

106,106′—fins

108—locking teeth

FIG. 11B

110—implant

111—teeth

112,112′—fins

114,114′—shelves

115,115′—chamfers

### DETAILED DESCRIPTION OF THE PREFERRED EMBODIMENTS

The implant itself is preferably allograft material but may also comprise a variety of presently acceptable biocompatible materials. The body of the implant may optionally have a modest taper to accommodate the natural lordosis of the lumbar spine. In a variant of one embodiment, locking notches or teeth may be located on the outer edge of both the stabilizing fins, to engage the cortical bone and prevent the implant from migrating out of the intervertebral space.

As previously noted, any of the embodiments of the interlocking implant can be fabricated from cadaver bone which is processed to form bone allograft material. Tissue grafting of living tissue from the same patient, including bone grafting, is well known. Tissue such as bone is removed from one part of a body (the donor site) and inserted into tissue in another (the host site) part of the same (or another) body. With respect to living bone tissue, it has been desirable in the past to be able to remove a piece of living tissue graft material which is the exact size and shape needed for the host site where it will be implanted, but it has proved very difficult to achieve this goal.

It is now possible to obtain allograft bone which has been processed to remove all living material which could present a tissue rejection problem or an infection problem. Such processed material retains much of the structural quality of the original living bone, rendering it osteoinductive. Moreover, it can be shaped according to known and new methods to attain enhanced structural behavior. In the present invention, allograft bone is reshaped into one of the spacer configurations for use as a spine implant.

In the current invention, a blank is cut from cortical allograft bone, generally from long bones of the leg. The blank is machined by conventional milling to form the fins, grooves and outer surfaces. Such processes in general are able to maintain the biological and structural properties of the allograft material.

FIGS. 3A, 3B, 3C and 7B depict the implant and its position once inserted within two vertebrae.

FIGS. 1A, 1B, 2, 4B, and 5A-6B depict the surgical tools used to install the implant. This apparatus comprises a set of unique tools which will accurately cut the rectangular grooves in bone and prepare the endplate surfaces for the purpose of inserting an implant which locks adjacent vertebrae together.

FIGS. 8 and 9A-9C depict the surgical tool used to facilitate translational and angular distraction from within the disk space to achieve the quality of distraction currently only obtained by the anterior approach. A double action vertebral spreader is used that will penetrate more deeply into the disk space to create anterior lift to a fixed or variable angle. The design will allow the surgeon to set the lordotic angle prior to distraction of the vertebral endplates.

FIGS. 10A-F and 11A and B depict the various embodiments of the spacer/osteotome guide and of the implant.

Overview of the Invention

Referring to FIGS. 1A through 3C there are shown the three main components of the present invention. FIG. 1A is an oblique view of a spacer/osteotome guide 2, two each of which are inserted between adjacent vertebra 12, 14 as shown in FIGS. 4A and 4B. FIG. 1B shows the angle α of the taper of the spacer, said taper corresponding to the desired lordosis of the two vertebrae being fused. FIG. 2 is an oblique view of a dual box osteotome 4 comprised of a two-part osteotome blade 6 and a driver handle 8. As will be explained below, the osteotome blade 6 is guided by the guide 2 when it cuts channels in adjacent vertebral endplates so as to accommodate the insertion of a spinal fusion implant 10 as shown in the three orthogonal views, FIGS. 3A, 3B and 3C. FIG. 3A shows curved sides 7a, 7b which correspond to the edges of the source bone from which the implant 10 has been machined, specifically from cortical bone of the femur or tibia. FIG. 3B is a side view showing the tapered side 7b (with corresponding taper on unshown side 7a), which has the same angle α that provides the desired angle or lordosis of the vertebrae being conjoined by the implants, specifically a lordotic angle of 2α or about 10 degrees. FIG. 3C shows the implant 10 from its posterior end, i.e., the end that, when installed corresponds to the posterior side of the spinal column. The serrations or locking teeth 11 provide a gripping effect when the implant has been installed in the channel that has been cut by the cutting tool 4, said channels being visible in FIG. 7A. During the implantation process, two implants are installed in each intervertebral space, more or less symmetrically about the spinal cord, as will be described in more detail below.

Use of the Invention

FIG. 4A is a posterior view of two vertebra 12, 14 from which boney, muscle-supporting processes have been removed to expose the spinal cord 16, the nerves 18a, 18b, 18c and 18d extending laterally outward therefrom, and the posterior portion of the intervertebral space 20, within which two implants 10 are to be inserted, one on either side of spinal cord 16 within the specific locations occupied by the spacer/osteotome guides 2 in FIG. 4B. Note that the respective vertebrae 12, 14 have been separated in FIG. 4B compared to FIG. 4A to accommodate the installation of the spacer/osteotome guides 2, or, more specifically, the space 20′ in FIG. 4B is larger than the corresponding space 20 in FIG. 4A.

The Spacer/Osteotome Guide

Referring to FIGS. 5A and 5B, the vertebral body spacer/osteotome guide 2 is shown in two oblique views, showing the length L, width W and height H dimensions. The spacer/osteotome guides 2 are made of stainless steel.

The spacer/osteotome guides 2 measure approximately 20-30 mm long by 9-12 mm wide, and they have heights that vary from 6 mm to 14 mm. The anterior or front part of the spacer is chamfered or curved to facilitate introduction past bony landmarks. The posterior end has features to allow connection to a drive handle, which is easily removed after the spacer is fully inserted into the disc space. There are two centrally located coplanar slots 34 on the superior and inferior surface of the spacers which are approximately 1 mm to 3 mm deep, defining a guide channel. The posterior origin of these slots 34 is easily viewed, even when the spacer is fully inserted. The spacers further have tabs 30 extending superiorly and inferiorly that contact the vertebral body's posterior wall to prevent over insertion. In a second embodiment, the spacers may have a centrally located, hollow tab projecting 2 mm to 5 mm posteriorly so as to guide the bone cutting tome blade on both sides and also to provide additional protection to adjacent neural structures. This second embodiment also includes a flange projection on one lateral side that, when fully inserted, retracts the central dura. The combination of the hollow tab and flange provide full protection while allowing the safe subsequent passage of the sharp bone-cutting tome, and prevents over-insertion.

FIGS. 5A and 5B show details of the spacer/osteotome guide 2 in two oblique views. FIG. 5A is a rear and side view of the spacer/osteotome guide 2, showing the aforementioned stop tabs 30 which are contiguous with the main body 32, and two osteotome guide channels 34. The hole 36 receives the end of a detachable handle, not shown, which is used to insert the spacer/osteotome guide 2 between adjacent vertebrae 12, 14 as shown in FIG. 4B. FIG. 5B is a partial front and side view of the spacer/osteotome guide 2. FIG. 5C is a schematic cross-sectional side view of a spacer/osteotome guide 2 within the vertebral space 20″ between two vertebrae 27, 28. FIG. 5C is a side view of one of the installed spacers/osteotome guides 2 with stop tabs 30 abutting the posterior side of an upper vertebra 27 and a lower vertebra 28. FIG. 5C complements FIG. 4B where the spacers/osteotome guides 2 are shown in posterior view between vertebrae designated as 12 and 14. Note that, as shown in FIG. 5A, the spacer/osteotome guide 2 has only three stops 30. The reason for only three stops 30 is evident in FIG. 4B where the nerves 18a, 18b are in proximity to where the missing fourth stop would otherwise be. Note yet further in FIG. 4B that the two spacers/osteotome guides 2 shown are not identical, but rather they are mirror images of each other with respect to the sagittal plane, or, in other words, in relation to the locations of the three respective stops tabs 30 on each spacer.

The Osteotome

The dual box osteotome 4, i.e., the osteotome, is shown in oblique views in FIGS. 2, 6A and 6B. The osteotome 4 is comprised of two parallel, hollow cutting blades 6 and a detachable handle 8. The double blade portion is further connected to a male or female threaded boss to enable firm attachment to the handle 8. Each box shape blade 6 is generally 4 mm wide by 4 mm tall on each side. Three sides of the box are sharpened and one side is blunt. The blunt side, generally the side closest to the central axis, may also protrude 1 mm to 3 mm from the sharp sides and may be chamfered. More specifically, as shown in FIG. 6A, the front sides 40 of the hollow cutting blades 6 have sharp cutting edges 42. FIG. 6B shows in oblique view the way in which the osteotome and handle assembly 4 engages the channels 34 in the spacer/osteotome guide 2. The arrow 45 shows the direction of the osteotome 4 when its blade portion 6 engages the spacer/osteotome guide 2 after the spacer/osteotome guide has been inserted between the vertebrae as shown in FIG. 4B. The cutting force to drive the cutting blade assembly 4 is applied by way of the handle 8, through the use of a mallet tapping against the end 15 of the handle portion 8 of the osteotome assembly 4 shown in FIG. 6A.

As the hollow cutting edges or blades 6 of the osteotome cut into the adjacent vertebral end plates 21, 23, the pieces of cut bone accumulate inside the hollow spaces 44 shown in FIG. 6B. The open ended design of the cutting blade 6 facilitates removal of the bone chips and later cleaning of the instrument 4.

The depth of cut of the cutting blade into the vertebral endplates is intended to be sufficient to remove the hard cortical bone of the endplates 21, 23 shown in FIG. 7A of the vertebral bodies so as to expose blood-rich, underlying cancellous bone. FIG. 7A shows, in a posterior view, the channels 50 that have been cut by the cutting blade assembly 4. The objective of the cutting process is to expose a significant bed of bleeding bone while maintaining a sufficient portion of strong cortical endplate bone.

The Implants

Two implants are used between each the vertebral bodies being fused. Each one is to provide structural support and stabilization to a lumbar spinal motion segment subsequent to removal of protruding or deranged intervertebral disc material, and also to provide a substrate for new bone growth accompanying successful fusion of two adjacent vertebral body segments.

Referring to FIGS. 3A through 3C, the implant 10 has fins 13 projecting inferiorly and superiorly from the central ⅓ of a wedge shaped block. When viewed from behind, as in FIG. 3C, the geometry of thc bone spacer 10 resembles a “cross”. When viewed from the side (FIG. 3B), the implant 10 is wedge shaped. Such that sides 7a, 7b, including the fins 13, diverge from the posterior side 10b to the anterior side 10a about a line of symmetry. The outermost finned surface has a series of locking teeth 11, or grooves or projections, that aid in anchoring the implant and its fins that engage the channels 50 shown in FIG. 7A. More specifically, the sharp, tooth—like projections 11 are about 1 mm tall, which is adequate to penetrate exposed cancellous bone after the vertebral endplate cortices have been cut to accommodate the fin portion 13 of the implant 10 thereby increasing interface friction and minimizing the potential for translation after implantation.

The importance of achieving good fit of a spacer 10 within the disc space is essential. When the fit is maximized the surface area of contact and resultant friction at the interface is maximized. Accordingly, for lordotic disc spaces the anterior height of the spacer device 10 is taller than the posterior height. The anterior region 10a has a slope or chamfer 9 to aid in initial insertion between the vertebrae. Alternatively, for parallel shaped disc spaces, a non-lordotic or parallel spacer (FIGS. 10C-10F) may provide a preferred fit.

The implant 10 shown in FIGS. 3A through 3C, is cut from human donor bone which accounts for the curved faces 7a, 7b which are most evident in the top view shown in FIG. 3A. More specifically, the implant 10 is cut from donor cortical bone of the femur or tibia. FIG. 7B shows two inserted implants 10 in posterior view between the vertebral bodies. FIG. 7C is a side view of an installed implant 10 within an intervertebral space 20 between two vertebrae 12, 14. The respective posterior—to—anterior angles α are shown in FIG. 3B, while the corresponding lordosis angles α are shown in FIG. 7C.

In FIG. 3B, the faces, or sides, 7a and 7b of the implant 10 are shown to be tapered at an angle α that corresponds to half of the desired lordosis of the vertebra. The angle α of the tapered side, shown in FIG. 3B corresponds to the angle α of the spacer/osteotome guide 2 shown in FIG. 1B. In FIG. 3C, the implant 10 has opposing slots 7c, 7d on either side to facilitate gripping with a holding instrument (not shown). The preferred configuration of the implant 10 is approximately 20 mm to 25 mm long by 9 mm to 12 mm wide by 6 mm to 14 mm high, as measured on the posterior region.

The Vertebral Body Spreader

FIG. 8 shows in oblique view the vertebral body spreader tool 60 used to separate or distract two adjacent vertebral bodies prior to insertion of the spacers 2. The spreader 60 enables sequential angular and translation distraction of the disc space from the posterior side of the spine.

More specifically, the spreader device 60 consists of two jaws 62, 62′ (which get inserted into an intervertebral disc space) connected to arms 64, 64′ each having a catch mechanism 65, 65′ that engages the respective locking stops 68, 68′. The arms 64, 64′ and the contiguous jaws 62,62′ pivot about the hinge pins 61,61′, respectively, so as to provide angular motion of the jaws. The handle grips 66, 66′ operate to displace the parallel rails 70, 70′ by way of the crossing slider mechanism 76, the operation of which is shown in FIGS. 9A through 9C. The crossing slider 76 consists of two arms 78, 79, which pivot about a common pivot point 80 when the handles 66, 66′ are squeezed together. Two ends of the crossing slider mechanism 76 engage respectively the handles 66, 66′ at the respective pivot points 77, 77′, which also slide forward (toward the jaws 62, 62′) inside of slots 82, 82′ in the rails 70, 70′ (visible in FIG. 8). The crossing slide mechanism maintains the rails 70, 70′ parallel with one another as they separate from one another when the handles 66, 66′ are squeezed together (FIG. 9C).

A design criterion of the vertebral body spreader 60 is to take into account a common characteristic of the degenerated painful disc, namely loss of disc height and loss of lordotic orientation. The goal is to restore natural height and angle to a collapsed disc space. Since the greatest degree of angular collapse is anterior, it is particularly difficult to lift the anterior portion of the disc space with a device that is applied from the posterior direction.

Spreaders of the sort typically used in posterior operations make contact only the posterior wall of the vertebral body and therefore provide only posterior lift. A consequence of posterior lift is anterior settling, resulting in a flattening of the disc space beyond anatomical norms. The spreader device 60 in FIG. 8 overcomes these disadvantages by way of two cooperating mechanisms that allow the following sequence of events: insertion, followed by angular distraction and then translational distraction. Referring now to FIGS. 9A through 9C, insertion of two opposed jaws 62, 62′ into the disc space 55 between two adjacent vertebral bodies 57, 59 is achieved when the device 60 is in the fully closed position, as shown in FIG. 9A. The length of the jaws 62, 62′ is 24 mm, which is sufficient to ensure that the jaws make good contact with the anterior portion of the disc space. The locking pivot arms 64, 64′ are then engaged into the position shown in FIG. 9B with the catches 65, 65′ seated in the respective locking stops 68, 68′, so as to angulate the jaws 62, 62′ at an approximately 10 degree angular distraction within the disc space 55. With the jaws locked at 10 degrees, a second translational motion is brought about by means of the handles 66, 66′ which cause parallel translational spreading of the disc space, as shown in FIG. 9C. This dual action ensures the disc space can be maintained at about 10 degrees while achieving maximum disc height restoration. Flattening of the disc height is prevented by maintaining of the 10 degree jaw position while distracting.

The spreader tool 60 may also be used to help facilitate insertion of the graft in the final stages of the operation. The dual action spreader 60 may be placed on the contralateral side of the disc space or directly adjacent to the graft if space is available, and it may be used to create additional lift and angulation, as required, to lessen the force required to insert the bone graft.

Summary of Operational Sequence

Referring to FIGS. 4A and 4B as well as FIGS. 7A through 7C, the installation of the implants 10 can be described in a general, summarizing way. First, the posterior faces of the vertebrae 12, 14 are exposed and then the vertebrae are forced apart to accommodate the insertion of the two spacer/osteotome guides 2, as shown in FIG. 4B. FIG. 6B shows the relationship of the spacer/osteotome guide 2 and the osteotome 4 when the cut is made to create each channel 50 as shown in FIG. 7A. The implants 10 are then installed as shown in FIGS. 7B and 7C.

More specifically, prior to implantation, the disc material is removed from the intervertebral space 20, shown in FIG. 4A, exposing the cortical endplates 21, 23 of the adjacent vertebral bodies 12, 14. The vertebral bodies are displaced from one another by use of the spreader tool 60 so that the intervertebral space 20 can receive the two rigid spacers/osteotome guides 2, shown in FIG. 4B, which are placed, one at a time, contralaterally in relation to the spinal cord 16. Each spacer/osteotome guide 2, upon being installed into the intervertebral space 20, maintains contact with the strong, cortical, endplate bone.

After the spacers/osteotome guides 2 are in place, the endplates 21, 23 are further prepared by inserting the dual box osteotome 4, FIG. 6A, that simultaneously removes from the respective top and bottom vertebral bodies (12, 14, respectively) a 4 mm wide by 4 mm deep portion of each endplate and vertebral body bone. The resultant channels 50 (FIG. 7A) in the respective vertebral bodies 12, 14 are parallel to each other in a plane that is parallel to the sagittal plane. The resultant channels 50 define a placement axis for the finned implant device 10.

The wedge-shaped implant 10, as shown in FIG. 3B, does not precisely match the geometry of the respective prepared bone slot in the endplates 21, 23. More specifically, the channels or slots 50 are cut such that they are parallel, not dependent on the planes of the respective vertebral endplates 21, 23. The maximum height of the fins on the anterior side 10a (FIG. 3B) is greater than the distance between the prepared slots 50. (As measured across the parallel bottoms of the slots.) Engagement of the leading chamfer 9 (FIG. 3B) into the prepared channels 50 locates the implant parallel to the sagittal plane. Subsequent impacting of the implant 10 causes the geometry of the initially parallel bottoms of the slots or channels 50 to assume the respective angles α of the implant fins 13 (carrying the teeth 11). Distraction of adjacent vertebral bodies through impaction upon the implant 10 is possible because the adjacent vertebral bodies 12, 14 are non-constrained. Their relative positions are controlled primarily by soft tissue structures (not shown) that can be non-destructively stretched or altered. A consequence of full device impaction is translational and angular distraction of the disc space 20 so as, in the end, to yield the lordosis angle 2α, shown in FIG. 7C/D.

The two fins 13 of each implant 10 are also slightly wider than the prepared channel 50 shown in FIG. 7A, creating a press fit when impacted. The tight fit achieves increased biomechanical stability and reduces the likelihood of migration of the implant after installation. Since the channels 50 in the vertebral endplates are only 4 mm wide, the endplate on either side of the channel retains its strength for good structural support.

Embodiments

FIGS. 10A through 10F show three embodiments of the spacer/osteotome guide. FIGS. 11A and 11B show two embodiments of the implant.

Spacer/Osteotome Guide—First Embodiment

FIGS. 10A and 10B show two views of the first embodiment of the spacer/osteotome guide 91. One of the distinguishing characteristics of this embodiment is the angle α made by the top T and bottom B relative to the central axis A-A′, the angle α being half the desired angle of lordosis, which is about 10 degrees. The other distinguishing feature is the stop tabs 92, located on the posterior end of the spacer block 91. The stop tabs 92 prohibit the spacer from moving to deep into the intervertebral space during insertion or during the bone cutting process. The hole 93 receives a detachable handle, used during insertion and removal of the spacer.

Spacer/Osteotome Guide—Second Embodiment

FIGS. 10C and 10D show two views of the second embodiment of the spacer/esteotome guide 94. Unlike the first embodiment above, the top T and bottom B of this embodiment are parallel, and no stop tabs are used. The hole 95 receives a detachable handle, used during insertion of the spacer and removal of the spacer. The blunt nose 94′, located on the anterior end, aids in the spacer insertion process.

Spacer/Osteotome Guide—Third Embodiment

FIGS. 10E and 10F show two views of the third embodiment of the spacer/osteotome guide 96. The main body 97 of the spacer is characterized by having a top T and bottom B that are parallel one another. The spacer is further characterized by an additional box guide 99 that receives the osteotome during bone cutting. The box guide 98 also serves to restrain the spacer block 96 from moving too deep into the intervertebral space during the bone cutting process. The hole 99 receives a detachable handle, used during insertion and removal of the spacer. The blunt nose 96′, located on the anterior end of the spacer, serves to distract the respective vertebral bodies during the spacer insertion process.

Implant—First Embodiment

FIG. 11A is an orthogonal side view of the first embodiment of the implant 102. The characterizing feature of this first embodiment is the angle α′ which each shelf 104, 104′ (there are two shelves on each side, the second set is out of view in the FIGURE) makes relative to the main axis B-B′. Each angle α′ is half the desired angle of lordosis, which is about 10 degrees. Chamfers 105, 105′, which aid in the insertion process, are located on the anterior end of the implant. The two fins 106, 106′, respectively at the top T and the bottom B and having locking teeth 108, each make an angle α″ relative to the axis B-B′. The angle α″ is essentially equal to the angle α′, both being about half the angle of lordosis, or about 10 degrees.

Implant—Second Embodiment

FIG. 11B is an orthogonal side view of the second embodiment of the implant 110. This second embodiment is characterized relative to the first embodiment in that the sets of teeth 111 located on the tops of the respective fins 112, 112′ are parallel to the main axis C-C′, whereas each shelf 114, 114′ (there are two shelves on each side, the second set is out of view in the FIGURE) makes an angle α′″ relative to the main axis C-C′. Each angle α′″ is half the desired angle of lordosis, which is about 10 degrees. Chamfers 115, 115′, located on the anterior end of the implant, aid in the insertion process.

While the invention has been described in combination with embodiments thereof, it is evident that many alternatives, modifications, and variations will be apparent to those skilled in the art in light of the foregoing teachings. Accordingly, the invention is intended to embrace all such alternatives, modifications and variations as fall within the spirit and scope of the appended claims.

We claim:

1. An implant for insertion into a space left by surgically removed spinal tissue, said implant comprising:
a. a body having a top surface and a bottom surface and a distal end and a proximal end; and
b. on each of the top surface and the bottom surface of the body, a stabilizing fin protrusion generally in the form of a rectangular parallelepiped, each said stabilizing fin protrusion having a height and width configured to slide matingly into a corresponding channel in an adjacent vertebra, said channels having been pre-established with substantially the same height and width as the stabilizing fin protrusion which is inserted into said channel, each said stabilizing fin protrusion further having a surface in a plane approximately perpendicular to the plane of the vertebral body endplate or disk after the implant is inserted into said space, whereby said implant attaches mechanically to the vertebrae adjacent to said space.

2. A matched pair of implants according to claim 1 in which each member of the matched pair is substantially a mirror image of the other member of the matched pair, each member of the matched pair being separately implanted on opposite sides of the spinal column.

3. The implant of claim 1 in which the body has the shape of a segment of an annulus.

4. The implant of claim 1 in which the surface of each said stabilizing fin protrusion in a plane approximately perpendicular to the axis of the spine additionally comprises locking teeth.

5. The implant of claim 1 in which the top surface and the bottom surface of the body have an angle with respect to one another which is predetermined to maintain after implantation the natural lordosis of the spinal segment in which the implant is being used.

6. The implant of claim 5 in which the surface of each said stabilizing fin protrusion in a plane approximately perpendicular to the axis of the spine have an angle with respect to one another which is predetermined to maintain after implantation the natural lordosis of the spinal segment in which the implant is being used.

7. A combined spacer and osteotome guide for maintaining a proper intervertebral space and for preparation of an adjacent vertebrae to receive stabilizing fin protrusions of an implant, said spacer and osteotome guide comprising:
a. a body generally in the shape of a rectangular parallelepiped and having a top surface and a bottom surface and a proximal end and a distal end;
b. tabs at the proximal end of the body to prevent inserting the spacer and osteotome guide too far into the intervertebral space; and
c. a guide channel in each of the top surface and the bottom surface matching in width and depth the outer dimensions of an osteotome to be inserted over the spacer and osteotome guide.

8. The spacer and osteotome guide of claim 7 in which the top surface and the bottom surface have an angle with respect to one another which is predetermined to maintain the natural

9. An osteotome comprising:
a. a handle; and
b. a pair of osteotome blades, each blade in the shape of a hollow elongated rectangular box, said boxes being rigidly attached to the handle, said osteotome blades being configured so as to slide matingly into a spacer and osteotome guide.

10. The osteotome of claim 9 in which the pair of hollow osteotome blades additionally comprises a structural member disposed transverse between the pair of hollow osteotome blades to comprise an assembly, said assembly comprising a dual box, said assembly further being rigidly attached to the handle.

11. A vertebral body spreader capable of producing sequential angular and translational distraction of a disk space comprising:
a. a pair of jaw actuation arms, each said arm having a jaw at the proximal end;
b. a crossing slider mechanism comprising two arms, the arms being pivotally connected at the mid point of each said arm;
c. a pair of parallel rails, each said rail being connected pivotally at one end to one of the jaw actuation arms so as to provide angular of the jaws and near the other end being connected both pivotally to the proximal end of one of the arms of the crossing slider mechanism and slidably connected to the distal end; and
d. a pair of handle grips pivotally connected near the proximal ends and each said handle grip being connected pivotally at the proximal end to the distal end of one of the arms of the crossing slider mechanism.
mechanism and slidably connected to the distal end; and
d. a pair of handle grips pivotally connected near the proximal ends and each said handle grip being connected pivotally at the proximal end to the distal end of one of the arms of the crossing slider mechanism.

13. The vertebral body spreader of claim 12 in which each of the jaws is about 24 mm in length.

14. The vertebral body spreader of claim 12 in which each of the jaw actuation arms has a catch mechanism at the distal end.

15. The vertebral body spreader of claim 14 in which the distal end of each of the parallel rails has a locking stop for engaging the catch mechanism on the jaw actuation arms.

16. The vertebral body spreader of claim 15 in which the jaws are at an angular distraction with respect to one another which is predetermined to maintain the natural lordosis of the spinal segment in which the implant is being used when the a catch mechanisms of the jaw actuation arms are engaged by the locking stops of the parallel rails.
